(19) United States
(12) Patent Application Publication
(10) Pub. No.: US 2003/0092187 A1
(43) Pub. Date: May 15, 2003

(54) Method of detecting the release of substances from cell organelles by means of flow cytometry

(76) Inventors: Karsten Stahnke, Guenzburg (DE); Hubert Hug, Lonsee (DE); Klaus-Michael Debatin, Ulm (DE)

Correspondence Address: HELLER EHRMAN WHITE & MCAULIFFE LLP, 1666 K STREET,NW, SUITE 300, WASHINGTON, DC, 20006, US

(21) Appl. No.: 09/987,206

(22) Filed: Nov. 13, 2001

(51) Int. Cl. G01N 33/48

(52) U.S. Cl. 436/63; 436/164; 436/172; 436/174; 435/4; 435/29

(57) ABSTRACT

The invention is directed to a method of detecting the release of substances from cell organelles by means of flow cytometry, comprising the steps of: fixing cells to be studied; permeabilizing the outer cell membrane; staining the cells with a fluorochrome compound which binds specifically to at least one substance to be studied; measuring the individual cells by means of flow cytometry while stimulating the fluorochrome compound; and determining the fluorescent strength of the cells.

The present invention is directed to a well-calculated detection of the release of substances from cell organelles by means of fluorescence flow cytometry.

Flow cytometry is a method by means of which it is possible to measure the fluorescence of individual cells labeled with a fluorochrome. A flow cytometer comprises a storage vessel containing a cell suspension of labeled cells, a dropping path which a thin droplet filament or line of droplets flowing out of an opening of the storage vessel must travel, a laser which irradiates the line of droplets and one or more measuring channels which can measure the emission fluorescence of the line of droplets. In this case, the production of the line of droplets is critical since the objective is to achieve individual droplets each containing only one cell. In this way it is possible to realize that the laser irradiates only one cell each so that the measuring channels measure the fluorescence of a single cell. A connected data processing equipment stores the individual measurements and can perform a statistical evaluation.

A principle of flow cytometry is the measurement of relative fluorescences by means of voltage-controlled photomultiplier tubes (PMTs) as measuring channels. Here, the fluorescence signal is shown on a logarithmic scale of 10⁰ (start of the 1^{st} decade) to 10⁴ (end of the fourth decade). Calibration to this range is made by means of negative and positive control standards which shall only have background fluorescence in one case and the maximum expectable fluorescence to be determined in the other case. The PMT voltages used in this connection depend on the employed flow cytometer and are adjusted by means of the respectively used control standard.

It is also possible to compensate the measured values electronically. Electronic compensation is a standard method of multicolor flow cytometry. Labeling of cells with two or more fluorochromes sometimes creates the problem that the overlap of the emission fluorescence spectra in the case of strong fluorescence signals leads to an irradiation of the adjacent fluorescent channel where as a result thereof a false positive signal occurs. It is corrected within the scope of electronic compensation by electronic subtraction for each individual measuring event by subtracting a percentage of the initial fluorescence from the false positive fluorescence.

Apoptosis is a biological process in which a series of genetically controlled events is triggered in cells, which effect the controlled death and the efficient elimination of the affected cells. A detection of apoptosis and apoptosis-regulating molecules is decisive for the diagnosis and therapy of various diseases such as autoimmune diseases, viral infections, neurodegenerative diseases, bone marrow insufficiency syndromes, leukemias and solid tumors.

Apoptosis detection is often made in connection with the testing of potentially effective cytostatic agents. Cytostatic agents induce apoptosis by activating essential elements of the apoptosis program. The effectiveness of cytostatic agents depends on the extent to which the molecular apoptosis regulators are activated, in particular on the extent to which mitochondria are activated for the release of pro-apoptotic signal molecules. In particular the release of cytochrome c from mitochondria of cells which become apoptotic is an important step in the cell death program signal sequence involved in most forms of cell death.

The prior art knows mainly two methods of detecting the mitochondrial cytochrome c release. For one thing, separation of cytosole and mitochondria can be made in a separating process and a subsequent detection can be performed using an immunoblot. The increase of a measuring signal in the cytosolic fraction and the decrease of the measuring signal in the mitochondrial fraction is in this case interpreted as a release of cytochrome c from the mitochondria. This method is costly and time-consuming, susceptible to faults and also requires a large amount (about 50 million) of cells. Such a solution is thus virtually only usable in basic research to prove the principle of mitochondrial release. Another drawback of this prior art method is that the cells used can only be studied jointly so that it is not possible to detect a cytochrome c release in only part or a subpopulation of the employed cells. Hence it is not possible either to detect the cytochrome c release, which lacks in the case of cytostatic agent resistance, in only a partial population of the studied cells.

The other known method consists of a immunohistochemical detection of the release by staining cytochrome c in the cell using suitably labeled antibodies and an evaluation by means of fluorescence microscopy. This method is also time-consuming, and the microscopic evaluation calls for a lot of experience and depends on the analyst. In contrast to the above method, it is possible to analyze individual cells, the amount of analyzed cells being considerably limited by the microscopic observation and the limited cell number on a slide.

Testing the resistance to cytostatic agents is carried out conventionally by the detection of growth inhibition, cell death or decrease of metabolic functions (e.g. the reduction of MTT (3-(4,5-dimethylthiazole-2-yl)-2,5-diphenyl-tetrazolium bromide) in cell cultures treated with cytostatic agents. Although the cytostatic resistance testing by means of an MTT assay shows rough correlations with the response of a chemotherapy, it is not suited to make predictions for individual patients on the effectivenes of individual drugs.

A detection of the cytochrome c release in primary human cells for the purpose of detecting a cytostatic effect is not known in the art, which is due to methodical difficulties in connection with the above outlined methods. The only possibility of detecting ex vivo mitochondrial changes after apoptosis induction in patient cells is the measurement of the mitochondrial membrane potential which is, however, only changed in part of the apoptosis forms but not in all cell types.

It was therefore the object of the present invention to provide a method by which a more reliable and differentiated detection of apoptosis signal molecules is easily possible in cells with activated cell death program.

This object is achieved by providing the method according to independent claim 1. Further advantageous embodiments, aspects and details of the present invention follow from the dependent claims and the description.

The invention is based on the principle that it is possible to detect specifically substances in cell organelles by suitable pre-treatment of cells using flow cytometry. The method is thus not only suited for the detection of apoptosis but can be used quite generally where substances which also occur or can also occur in the cytoplasm and would otherwise be measured as well, shall be detected in organelles.

Before the present methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, “and” “the” include plural reference unless the context clearly dictates otherwise.

Thus, for example, reference to “a host cell” includes a plurality of such host cells, reference to the “antibody” is a reference to one or more antibodies and equivalents thereof known to those.skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the methodologies which are reported in the publications which might be used in connection with the invention. Nothing herein is to be construed as an admission that the invention is not entitled to antedate such disclosure by virtue of prior invention.

The invention is thus directed to a method of detecting substances released from cell organelles by means of flow cytometry, comprising the steps of:

fixing cells to be studied,

permeabilizing the outer membrane thereby obtaining the organelle structure;

staining the cells with a fluorochrome compound which binds specifically to at least one substance to be detected; and

measuring the individual cells by means of flow cytometry while stimulating the fluorochrome compound; and

determining the fluorescent strength of the cells.

By this method it is possible to remove the substances to be detected from the cytosole, if they were located there, since the outer cell membrane has become permeable, however, the substances stay in the cell organelles since they are bound to organelle structures there and hence cannot leave the cell. Substance still present in the organelles can then be determined by attachment of a fluorochrome compound by means of flow cytometry.

According to the invention cells are understood to mean all animal or human cells which have an activated cell death program. These are preferably primary tumor or leukemia cells.

Within the meaning of the present invention a substance to be detected is any chemical substance present in the cell, which can be detected by means of a fluorescence compound, e.g. by direct or indirect binding etc. It may comprise nucleic acids such as DNA, RNA, t-RNA, as genomic or plasmid DNA, mRNA, mitochondrial DNA etc. or proteins, peptides and protein complexes or other molecules such as metabolic products, structural components of the cell of carbohydrate polymers, membrane components or other cell components.

The invention shall by no means be limited to cytochrome c even though the desire for its simple detection is described as a starting base of the invention. A person skilled in the art can rather infer from the description of the invention that the invention is applicable to a number of compound classes and opens up a plurality of fields of application. Such an application is e.g. the release detection of other apoptosis signal molecules from mitochondria, such as Smac or AIF (apoptosis inducing factor), or from the endoplasmic reticulum, such as caspase 12.

After the flow cytometry which is always computer-assisted, a data record on the individual measured cells is available, which can be processed and interpreted as usual and by means of corresponding interpretive programs. The method according to the invention may comprise e.g. the further step of

calculating the cell portion above and/or below a given limiting fluorescence value.

This embodiment serves for reducing the resulting primary data material to a simpler yes/no decision by determining limiting values, i.e. for answering the question of whether the substance searched for is present in an organelle or in the cytoplasm. The person skilled in the art knows how to determine corresponding limiting values and such determinations can be made in preliminary tests.

In order to accelerate the diffusion of substances, namely the substance to be studied, from the cytoplasm of the fixed cells, it can also be advantageous to wash the cells after the permeabilization with a wash solution to remove the substance to be detected from the cytoplasm of the cells. Examples of suitable wash buffers are saponin (0.1%), digitonin (0.1%), triton ×100 (0.01%). It can also be advantageous to increase the binding of the substance to the wash solution by adding proteins and selecting a suitable pH value.

A cell organelle within the meaning of the present invention is understood to mean all compartments inside of cells which are separated by their two-layered membrane from other compartments of the cell such that the substance to be studied cannot reach the cytosole without a permeabilization of the compartment membrane. For example, the cell organelles may be mitochondria, chloroplasts, Golgi systems, endoplasmic reticulum or cell nuclei. Depending on the permeability of the inspected organelle membrane different substances can be studied, if desired, e.g. only substances which have more than one limiting molecular mass for the passage through the organelle membrane.

The first step of the method according to the invention is the fixing of the cells to be investigated. By this, their shape is stabilized and they are more easily accessible to the subsequent steps. Fixing can be carried out as usual with known fixing agents in common methods.

It is preferred to use paraformaldehyde, glutaraldehyde or methanol for fixing.

A central step of the method according to the invention is the permeabilization of the outer cell membrane to enable the substance to be studied to leave the cytosole. It is only possible by permeabilization to make a distinction as regards the localization of the substance to be detected. In so far as the substance to be studied is located in one of the organelles, washing-out after the permeabilization of the outer cell wall is not possible, so that after the fluorochrome binding the substance can still be detected by means of flow cytometry. On the other hand, if the substance can be detected in the cytoplasm, it will be diffused out of the cell by permeabilization so that detection by means of flow cytometry is no longer possible or the measured signal is considerably smaller. The outer cell membrane is preferably permeabilized by means of a detergent. However, it is also possible to use other techniques of permeabilization, e.g. a treatment with hypotonic solutions. The employed detergent can be selected from saponin (0.1%), digitonin (0.1%) or triton ×100 (0.01%).

The fluorochrome compound used should have two functionalities to be suitable for the method according to the invention. On the one hand, it should contain an actual fluorochrome producing a fluorescence emission after stimulation in the flow cytometer. On the other hand, the fluorochrome compound should contain a substance linked with the actual fluorochrome and binding specifically to the substance to be studied. The employed fluorochrome depends on the respectively concrete wording of the question and the available fluorochromes. Preferred fluorochromes are fluorescein isothiocyanate (FITC), R-phycoerythrin, allophycocyanine or peridinchlorophyll. The person skilled in the art is familiar with carrying out suitable intracellular fluorescence stainings. A wide range of compounds which are known from the prior art for a plurality of substances is also available for the specific binding component. Here, nucleic acids and proteins proved particularly flexible when used, since they can be adapted to a number of substances to be studied. Nucleic acids are particularly suitable for binding to other nucleic acids by a complementary sequence of nucleotides. In particular, it is preferred for the fluorochrome compound to have a staining portion and a protein portion, the protein portion being preferably an antibody, antibody derivative or antibody fragment. By means of antibodies it is possible to detect a large number of substances to be studied with high specificity, e.g. other proteins, nucleic acids or structural elements within the cell.

The method according to the invention can be applied in particularly advantageous manner when the substance to be detected is a protein.

In a particularly preferred embodiment the substance to be detected is cytochrome c, so that unexpectedly this method is even usable for the detection of apoptosis and the cytostatic effectiveness or resistance.

It is particularly preferred to detect the presence of cytochrome c by binding to an anti-cytochrome c antibody (company BD Pharmingen) which in turn is detected by an FITC-labeled anti-mouse immunoglobulin antibody.

As explained already a particularly interesting field of application of the present invention is the observation of dynamic processes within the cell, e.g. the effect of cytostatic agents on the cell population. Therefore, it is particularly preferred for the cells to be incubated with an active substance prior to fixing to thus provoke a response to be studied.

Such an active substance is preferably a potential cytostatic agent whose apoptosis-inducing properties shall be determined.

However, it is also possible to incubate a known cytostatic agent with tumor or leukemia cells of a patient to determine via the method according to the invention the sensitivity of the malignant cells to various cytostatic agents. Furthermore, there is the possibility of studying cells from a patient who is undergoing a cytostatic agent therapy to find out whether this patient responds to the therapy (ex vivo monitoring of cytostatic agent therapy).

The method according to the invention has been described with respect to its isolating quality thus far. However, it is also possible to carry out more than one measurement by means of a flow cytometer at the same time. The simplest procedure is here the use of a flow cytometer having two or more lasers each emitting light in differing wavelength ranges and thus being able to stimulate different fluorochromes to emit fluorescence.

It is thus preferred according to the invention to determine simultaneously a second property of the cells to be studied by means of a second fluorochrome compound.

However, it is also possible to use a single-laser flow cytometer to measure two different fluorochrome compounds at the same time. In this preferred embodiment the invention is characterized in that the method is carried out with a single-laser flow cytometer having two fluorescence channels, the second fluorochrome having an emission wavelength range overlapping the first fluorochrome and the photomultiplier tube voltage of the fluorescence channel provided for measuring the second fluorochrome being adjusted such that no fluorescence emission signal produced in this fluorescence channel by the first fluorochrome is detected.

This preferred embodiment of the present invention is specified in German patent application DE 100 53 747.2 (herein incorporated by reference), so that a detailed description can be dispensed with herein.

In a preferred embodiment of the present invention the method according to the invention permits, e.g. in a simple and cost-saving manner, the measurement of the most important apoptosis-inducing mitochondrial signal process in combination with the simultaneous detection of caspase activation by means of conventional flow cytometry. Thus, it is possible for the first time to study two apoptosis signal paths in one cell by means of flow cytometry.

The established method of flow cytometry thus serves for studying the mitochondrial apoptosis signal path in connection with further biological parameters such as cell differentiation, cell cycle, cell activation, etc. The combined flow cytometric study with other apoptosis-regulating molecules enables another characterization of signal paths and their dependencies. By means of the connection with the detection of phosphatidylserine externalization using annexin V, the method according to the invention also enables comprehensive monitoring covering the wide range from the early apoptosis steps of mitochondrial activation to a late step of membrane modification. This detection is based on the principle that phosphatidyl serine is located in live cells exclusively on the inner side of the cell membrane. Following membrane modifications caused by the apoptotic process, phosphatidyl serine appears on the outer side of the membrane. Annexin V binds specifically to phosphatidyl serine so that the apoptotic membrane modifications can be detected by means of (FITC)-labeled annexin V.

For a further development of cytostatic agents it is decisive to identify substances which activate effectively mitochondrial signal paths since safe apoptosis induction can be assumed only in this case. By the use of flow cytometry the method according to the invention is particularly suitable for a preclinical testing regarding the effectiveness of newly developed cytostatic agents in established cell culture systems by means of the high-throughput method.

The method according to the invention also allows to measure the mitochondrial cytochrome c release together with the expression of surface epitopes. Thus, it is e.g. possible to identify leukemia cells in heterogenous cell populations, such as peripheral blood, and their chemosensitivity. In addition, resistant subpopulations can be further characterized according to their differentiation degree.

The decisive advantage of the method according to the invention are the low costs, the simple and rapid performance and the possibility of using currently available routine flow cytometers for the measurement. This ensures that the method will spread quickly as a diagnostic application e.g. for the chemosensitivity measurement in the case of leukemias and tumors.

The invention is further described by means of the figures in which

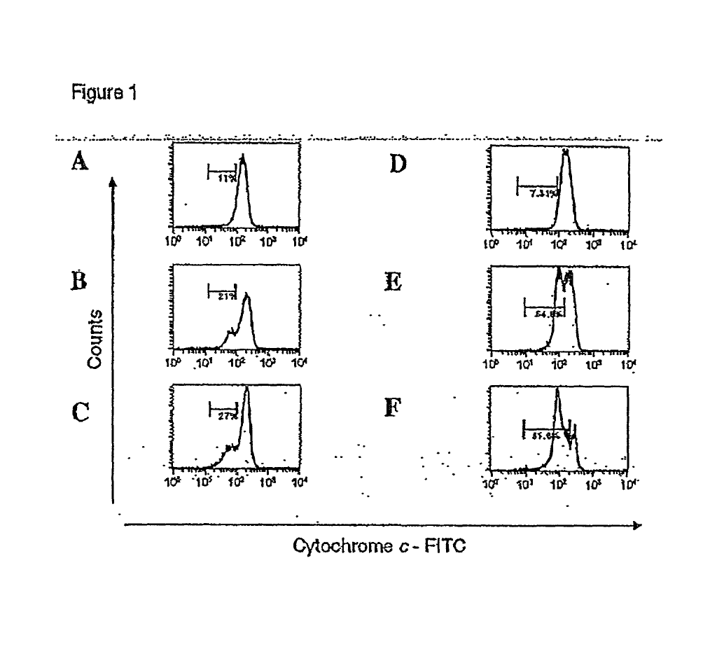

FIG. 1 shows an analysis of the mitochondrial cytochrome c release in CD95 and etoposide-induced apoptosis by flow cytometry Jurkat cells were cultured in medium alone (A, D), with cell death-triggering anti-APO-1 antibody for 3 hours (B) and 6 hours (C) or with etoposide for 4 hours (E) and 8 hours (F). Intracellular cytochrome c was detected by flow cytometry in fixed (4% PFA) and permeabilized (saponin 0.2%) cells. The fluorescence profile of the cytochrome c signal is depicted in the histogram. Percentages represent the proportion of cells with lowered cytochrome content.

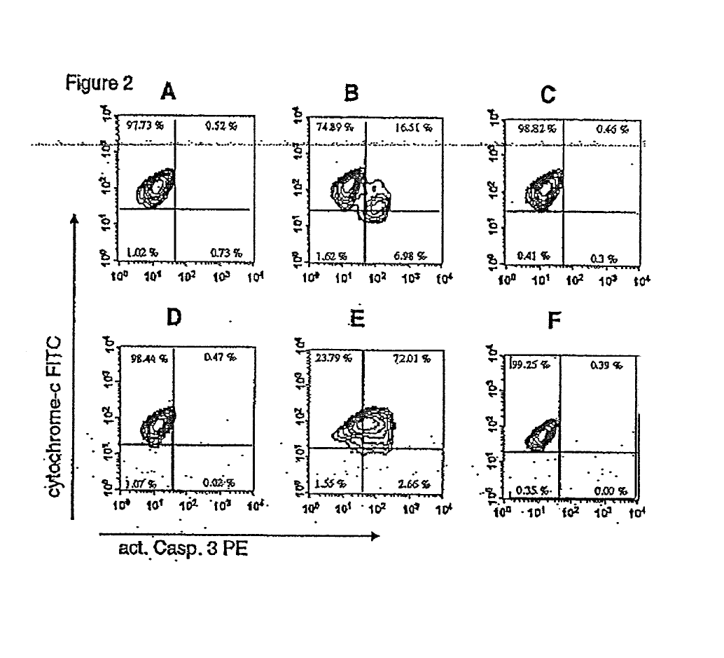

FIG. 2 shows the identification of deficient cytochrome c release and caspase-3 activation in cells transfected with Bcl-2

Jurkat cells (A-C) and Jurkat cells transfected with Bcl-2 (D-F) were incubated in medium alone (A, F), with anti-Apo-1 (B,E) and anti-APO-1 and the caspase inhibitor z-VAD-fmk (C, F) for 6 hours (A-C) or 24 hours (D-F). Live cells were identified in the forward/side scatter plot and analyzed for cytochrome c and active caspase-3 fragment. The figure depicts cytochrome c versus caspase-3.

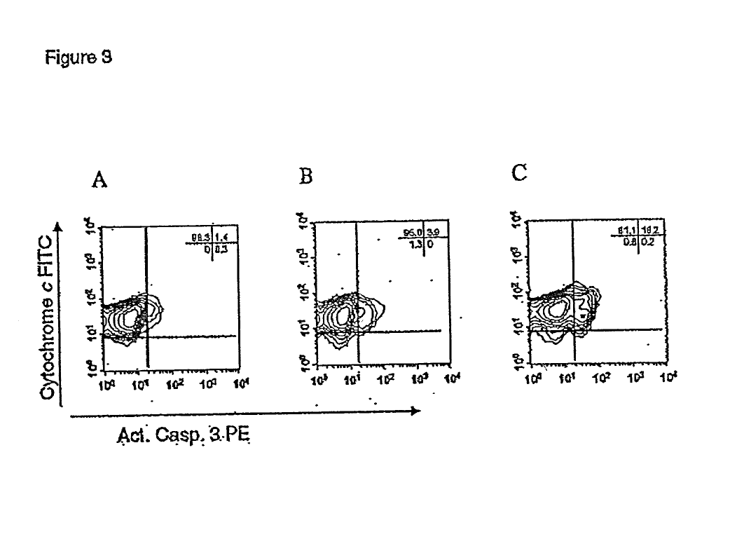

FIG. 3 shows the analysis of cytochrome c release and caspase-3 activation in primary leukemia cells of a patient with resistant disease

Leukemia cells of a patient with a myelocytic leukemia (FAB MO) resistant to chemotherapy were incubated in RPMI 1640 medium alone (A), etoposide 10 μg/ml (B) and OH-cyclophosphamide 3 μg/ml (C) for 24 hours. Live cells were identified in the forward/side scatter plot and analyzed for cytochrome c and active caspase-3 fragment. The figure depicts cytochrome c versus caspase-3. Note caspase activation in the absence of cytochrome c release in this leukemia sample.

The invention is described in more detail below by means of concrete embodiments.

### EXAMPLE 1

General Remarks:

Cell cultures

The Jurkat human T lymphocytic cell line was cultured in RPMI 1640 (Life Technologies, Inc., Eggenstein, Germany) with 10% fetal calf serum (FCS, Sigma Chemicals, Deisenhofen, Germany) together with penicillin, streptomycin and glutamine (Biochrome KG, Berlin, Germany) at 37° C. in humified air/5% CO₂. The vector control or Bcl-2 overexpressing SKW cell lines were maintained in RPMI 1640 (Life Technologies) containing 0.5 mg/ml geneticin (Life Technologies). 0.5×10⁵ cells/ml were cultured in 24-well plates for determination of apoptosis or in 75-cm² flasks (Falcon, Heidelberg, Germany) for protein isolation, microscopy or for flow cytometry.

Samples:

Leukemia blasts were isolated from bone marrow aspirates of leukemia patients by Ficoll-Hypaque density gradient centrifugation (Ficoll, density 1.077 g/l, Biochrome KG) at 300×g for 25 min. at 20° C., washed twice with Hanks salt solution supplemented with 2% fetal calf serum (HBSS/FCS) at 4° C., and adjusted to 10⁷ cells/ml. 1×10⁶ cells/ml were cultured in 24-well plates for determination of apoptosis for flow cytometry.

### EXAMPLE 2

Analysis of Leukemia Cell Populations

Cell analysis was performed by flow cytometry on a FACSCalibur® Cytometer (Becton Dickinson) equipped with a 488 nm argon and a 650 nm red diode laser. 0.5-1×10⁶ cells per test were stained for 25 min. at 4° C. with a combination of fluorochrome CD34PerCP, CD33APC, CD19APC conjugated to monoclonal antibodies for CD34, CD33 in the case of ALM, and CD34, CD19 in the case of ALL. The antibodies CD34 PerCp, CD33 APC were purchased from Pharmingen, CD19 was obtained from Coulter/Immunotech (Krefeld, Germany). For each antibody, an isotype-matched immunoglobulin was used as control in all experiments.

Samples were washed with HANKS salt solution supplemented with 2% bovine serum albumin (Serva company) and 0.2% azide (Merck, Darmstadt, Germany). After fixation with 2% paraformaldehyde the samples were immediately analyzed on a FACSCalibur® Cytometer. A minimum of 50,000 events per sample was acquired, stored in listmode files and subsequently analyzed with Cellquest® software (Becton Dickinson). Leukemia cells identified by CD34 CD33 co-expression were investigated for cytochrome c release and caspase activation.

The results are shown in FIG. 3.

### EXAMPLE 3

Analysis of Cytochrome c Release

The cell analysis was performed by flow cytometry on a FACSCalibur® Cytometer (Becton Dickinson) equipped with a 488 nm argon a 650 nm red diode laser. 1×10⁶ cells per test were washed with HANKS salt solution supplemented with 2% bovine serum albumin (Serva) and 0.2% azide (Merck) followed by a 20 min. PFA-solution (4%) fixation step. The cells were permeabilized with 0.2% saponin for 5 min. at room temperature. Unspecific binding of the anti-cytochrome c antibody was blocked with 5 μg mouse IgG1 (Sigma). Anti-cytochrome c antibody (7H8.2C12, Pharmingen) and anti-caspase-3 antibody (Beckton Dickinson) were added for additional 20 min. at 4° C. For cytochrome c staining the cells were incubated with goat anti-mouse IgG2b FITC (1:20, Southern Biotechnology Associates, Birmingham, Ala., U.S.). For each antibody, an isotype-matched immunoglobulin was used as control in all experiments. After fixation with 2% paraformaldehyde the samples were immediately analyzed on a FACSCalibur® Cytometer. A minimum of 50,000 events per sample was acquired, stored in listmode files and subsequently analyzed with Cellquest® software (Becton Dickinson).

The results are shown in FIGS. 1, 2 and 3.

1. A method of detecting the release of substances from cell organelles by means of flow cytometry, comprising the steps of:
fixing cells to be studied;
permeabilizing the outer membrane thereby obtaining the organelle structure;
staining the cells with a fluorochrome compound which binds specifically to at least one substance to be detected;
measuring the individual cells by means of flow cytometry while stimulating the fluorochrome compound; and
determining the fluorescent strength of the cells:

2. The method according to claim 1, characterized in that it comprises the further step of:
calculating a portion of cells which is above and/or below a given limiting fluorescence value.

3. The method according to claim 1 or 2, characterized in that following permeabilization the cells are washed with a wash solution to remove the substance to be detected from the cytoplasm of the cells.

4. The method according to any of claims 1 to 3, characterized in that the cell organelles are mitochondria, chloroplasts, Golgi systems, endoplasmic reticulum or cell nuclei.

5. The method according to any of claims 1 to 4, characterized in that fixing is made by paraformaldehyde, glutaraldehyde or methanol.

6. The method according to any of claims 1 to 5, characterized in that the outer cell membrane is permeabilized by means of a detergent.

7. The method according to claim 6, characterized in that the detergent is selected from triton ×-100, digitonin or saponin.

8. The method according to any of claims 1 to 7, characterized in that the fluorochrome compound has a dye portion and protein portion.

9. The method according to claim 8, characterized in that the protein portion is an antibody, antibody derivative or antibody fragment.

10. The method according to any of claims 1 to 9, characterized in that the substance to be detected is a protein.

11. The method according to claim 10, characterized in that the substance to be detected is cytochrome c.

12. The method according to any of claims 1 to 11, characterized in that prior to fixing the cells are incubated with an active substance.

13. The method according to claim 12, characterized in that the active substance is a potential cytostatic agent whose apoptosis-triggering properties shall be determined.

14. The method according to claim 12, characterized in that the active substance is a known cytostatic agent, whose resistance potential in cells shall be determined.

15. The method according to any of claims 1 to 14, characterized in that a second property of the cells to be studied is simultaneously determined by means of a second fluorochrome compound.

16. The method according to claim 15, characterized in that the method is carried out with a single-laser flow cytometer having two fluorescence channels, the second fluorochrome having an emission wavelength range overlapping the first fluorochrome and the photomultiplier tube voltage of the fluorescence channel provided for measuring the second fluorochrome is adjusted such that no fluorescence emission signal produced by the first fluorochrome in this fluorescence channel is detected.

17. The method according to any of claims 1 to 15, wherein the cells are primary tumor or leukemia cells.
